(19) United States
(12) Patent Application Publication
(10) Pub. No.: US 2017/0056341 A1
(43) Pub. Date: Mar. 2, 2017

(54) Extended Release Dosage Form Comprising Cyclobenzaprine Hydrochloride

(71) Applicant: Apotex Technologies Inc., Toronto (CA)

(72) Inventor: Bernard Charles Sherman, Toronto (CA)

(21) Appl. No.: 15/049,827

(22) Filed: Feb. 22, 2016

Related U.S. Application Data
(63) Continuation of application No. 14/840,644, filed on Aug. 31, 2015.

(51) Int. Cl. A61K 31/135; A61J 3/10; A61K 9/20; A61K 9/48; A61K 9/00

(52) U.S. Cl. CPC A61K 31/135; A61K 9/4808; A61K 9/0053; A61K 9/2054; A61K 9/2009; A61K 9/2013; A61J 3/10; A61K 9/4833; A61K 9/2095

(57) ABSTRACT

An improved extended release capsule of cyclobenzaprine, and in particular, cyclobenzaprine hydrochloride, is provided. The improved capsule, comprising one or more matrix-type tablets providing extended release of the cyclobenzaprine, provides a dosage form that is bioequivalent to the currently marketed AMRIX® capsules while providing a simplified manufacturing process. Also provided is a method for the preparation of the improved extended release capsule of cyclobenzaprine.

### CROSS-REFERENCE TO A RELATED APPLICATION

This application is a continuation of U.S. application Ser. No. 14/840,644, filed Aug. 31, 2105, the disclosure of which is incorporated herein by reference in its entirety.

### FIELD OF THE INVENTION

The present invention relates to an improved extended release pharmaceutical dosage form of cyclobenzaprine comprised of a capsule that is filled with one or more tablets providing the extended release of cyclobenzaprine or a pharmaceutically acceptable salt thereof.

### BACKGROUND OF THE INVENTION

Cyclobenzaprine hydrochloride is a skeletal muscle relaxant that is sold in the United States in 5 mg and 10 mg strength immediate release tablets, and in 15 mg and 30 mg strength extended release capsules.

Extended release capsules of cyclobenzaprine are sold in the United States under the tradename AMRIX®, and are made according to the teaching of U.S. Pat. No. 7,387,793. As described in U.S. Pat. No. 7,387,793, the extended release capsules were designed to provide a dosage form that enables once daily dosing, and that maintain an adequate plasma concentration-time profile to provide relief of muscle spasm associated with painful musculoskeletal conditions over a twenty-four hour period. This objective was achieved by preparing capsules containing a multitude of beads, with each bead consisting of a core comprising cyclobenzaprine hydrochloride, and an extended release coating comprising a water insoluble polymer membrane surrounding said core. Alternatively, the beads may be comprised of an inert core that is coated with a cyclobenzaprine layer and then overcoated with an extended-release layer.

The capsule of U.S. Pat. No. 7,387,793, when dissolution tested using USP (United States Pharmacopeia) Apparatus No. 2 (stir rate of 50 rpm, in 900 mL of a 0.1N HCl dissolution medium at 37° C.) exhibits a release profile substantially corresponding to the following pattern:
After 2 hours, no more than about 40% by weight of the total cyclobenzaprine is released from the beads;
After 4 hours, from about 40-65% by weight of the total cyclobenzaprine is released from the beads; and
After 8 hours, from about 60-85% by weight of the total cyclobenzaprine is released from the beads.

However, the process of manufacture of multi-particulate dosage forms according to the teachings of U.S. Pat. No. 7,387,793 is relatively complex. For example, core beads comprising an active ingredient are usually made by mixing the active ingredient with a polymer and water, extruding the material to produce strands, spheronizing the strands to produce beads, and then drying the resulting beads. The dried beads must then be coated with the extended release coating to provide the required extended release property to the beads. If inert cores are used, the manufacturing process is further complicated by the need to add both immediate-release and extended release coating layers. Similar extended-release compositions of cyclobenzaprine are described in U.S. Pat. No. 8,137,734.

A further approach for the preparation of extended-release compositions of cyclobenzaprine is described in WO 2012/042314, which is directed to the use of cyclobenzaprine in the treatment of tinnitus and other auditory dysfunctions. In this approach, the extended release composition is provided in the form of a polymer coating or matrix that comprises a water-insoluble polymer. These formulations are stated to combine the efficacy of cyclobenzaprine for treating tinnitus with an improvement in the desired relatively uniform plasma concentrations leading to maximum efficacy while reducing side effects and minimizing intersubject variability. While WO 2012/042314 describes formulations of cyclobenzaprine in general terms, WO 2012/042314 does not contain any example of the preparation of new cyclobenzaprine formulations, and instead relies on the use of AMRIX® capsules in the clinical trials that are reported therein.

WO 2012/122193 describes methods and compositions for treating depression using cyclobenzaprine. Pharmaceutical compositions providing controlled release of cyclobenzaprine are described as including a “controlled-release component” such as a lipid or mixture of lipids, liposome and/or microsphere that induce the controlled-release of cyclobenzaprine. Such dosage forms are exemplified in the form of a gelcap formulation using a mixture of lipids to form micelles containing cyclobenzaprine. Also, WO 2012/122193 does not describe capsule formulations that maintain the properties of the commercially available AMRIX® product in a dosage form with a simplified manufacturing process.

WO 2013/158638 describes a stable dosage form of skeletal muscle relaxants, one of which is cyclobenzaprine, with an extended release coating that is substantially free of plasticizer. More particularly, the invention of WO 2013/158638 is described as a multi-particulate pharmaceutical dosage form comprising extended release beads with active-containing core particles coated with an extended release coating surrounding the core particles. Thus, like the dosage forms of U.S. Pat. No. 7,387,793, the dosage forms of WO 2013/158638 involve a complex manufacturing process.

Published United States Patent Application No. 2012/0064164 describes an extended release cyclobenzaprine composition comprising cyclobenzaprine and a structured design with a core and an extended release coating where the cyclobenzaprine can be in the core, in the extended release coating or in between the core and the extended release coating. Thus, like the dosage forms of U.S. Pat. No. 7,387,793, the dosage forms of US 2012/0064164 involve a complex manufacturing process.

The prior art also discloses many pharmaceutical tablet formulations wherein the tablet is prepared as a matrix comprising a mixture of the active ingredient and one or more extended-release polymers that are either water-insoluble or form a viscous gel in water. The extended-release polymers serve to control the rate of release of the active ingredient from the tablet in gastrointestinal fluids. If the tablets are of large enough size (for example, greater than about 100 mg in weight), the rate of release of the active ingredient from the matrix may be adequately controlled without the need to apply an extended release coating to the tablets. However, for tablets small enough to be used to fill gelatin capsules (for example, tablets with a size of less than about 100 mg in weight), and particularly when the active ingredient is highly soluble in water, it is difficult to formulate a matrix-type tablet that can adequately extend release without the inclusion of an extended release coating.

Hence, one objective of the present invention to enable production of extended release capsules that are bioequivalent to AMRIX®, but made by a simpler, and more convenient to use manufacturing process.

### SUMMARY OF THE INVENTION

The present invention provides novel extended-release cyclobenzaprine hydrochloride capsules for once daily oral administration. Preferably, the capsules are bioequivalent to the currently marketed AMRIX® capsules while providing a simplified and more convenient manufacturing process. Thus, the present invention provides novel extended-release cyclobenzaprine hydrochloride capsules made using a manufacturing process that avoids many of the problems associated with the preparation of coated beads used in the currently marketed AMRIX® capsules.

It has been surprisingly found that, for cyclobenzaprine hydrochloride, despite its relatively high water solubility, it is possible to produce matrix-type tablets of small enough size to be used to fill gelatin capsules that provide satisfactory extended release of the active ingredient without the need to apply an extended release coating.

Accordingly, in one aspect of the present invention, there is provided an extended-release composition of cyclobenzaprine for oral administration in the form of a capsule containing one or more tablets, wherein each of the one or more tablets is a matrix-type tablet that comprises cyclobenzaprine, or a pharmaceutically acceptable salt thereof, one or more polymers, and optionally, one or more other pharmaceutically acceptable excipients, that provides extended release in an aqueous media without an extended release coating on the tablets;

wherein the capsule, when dissolution tested using USP Apparatus #2, complies with the following specifications:
(1) after 2 hours in 900 mL of 0.1 N HCl at 37° C. at 50 rpm, no more than about 40% of the cyclobenzaprine, or pharmaceutically acceptable salt thereof, is released from the composition; and
(2) after 8 hours in 900 mL of 0.05 M phosphate buffer at pH 6.5, 37° C. and 75 rpm, at least about 60% of the cyclobenzaprine, or pharmaceutically acceptable salt thereof, is released from the composition.

In a preferred embodiment of the composition, the one or more polymers comprises at least one polymer that is either water-insoluble or is an enteric polymer, and preferably at least one polymer is an enteric polymer. A preferred enteric polymer is hydroxypropyl methylcellulose acetate succinate, which is optionally a mixture of low and high pH-dissolving hydroxypropyl methylcellulose acetate succinate.

In a further preferred embodiment of the composition, the one or more tablets comprise from about 10% to about 60% cyclobenzaprine, or a pharmaceutically acceptable salt thereof, by weight, and from about 20% to about 80% by weight of the one or more polymers. More preferably, the tablets comprise from about 25% to about 50% by weight cyclobenzaprine, or a pharmaceutically acceptable salt thereof, and from about 40% to about 70% by weight hydroxypropyl methylcellulose acetate succinate. Even more preferably, the tablets comprise from about 25% to about 50% by weight cyclobenzaprine, or a pharmaceutically acceptable salt thereof, and from about 40% to about 70% by weight of a mixture of low and high pH-dissolving hydroxypropyl methylcellulose acetate succinate.

In a further preferred embodiment of the composition, the cyclobenzaprine is present in the form of its hydrochloride salt.

In a further preferred embodiment of the composition, the weight of each tablet is from about 5 mg to about 50 mg, and more preferably, from about 8 mg to about 40 mg.

In a further preferred embodiment of the composition, the total amount of cyclobenzaprine hydrochloride in the capsule is about 15 mg or about 30 mg. In a more preferred embodiment of the composition, the total amount of cyclobenzaprine hydrochloride in the capsule is 15 mg or 30 mg.

Preferably, the total amount of cyclobenzaprine hydrochloride in each of the one or more tablets is from about 2.5 mg to about 15 mg, and more preferably, the total amount of cyclobenzaprine hydrochloride in each of the one or more tablets is 15 mg, 7.5 mg, 5 mg, 3.75 mg, 3 mg or 2.5 mg.

In a further preferred embodiment of the composition, the one or more tablets are comprised of about 35% to about 40% by weight cyclobenzaprine hydrochloride, about 55% to about 70% by weight hydroxypropyl methylcellulose acetate succinate, about 0.3 to about 0.9% by weight magnesium stearate, and about 0.05 to about 0.5% by weight colloidal silicon dioxide. More preferably, the one or more tablets are comprised of about 37.5% by weight cyclobenzaprine hydrochloride, about 61.7% by weight hydroxypropyl methylcellulose acetate succinate, about 0.6% by weight magnesium stearate, and about 0.2% by weight colloidal silicon dioxide. Even more preferably, the hydroxypropyl methylcellulose acetate succinate is a mixture of hydroxypropyl methylcellulose acetate succinate grades wherein one grade of hydroxypropyl methylcellulose acetate succinate is soluble in aqueous solutions with a pH of about 5.5 and greater, and a second grade of hydroxypropyl methylcellulose acetate succinate is soluble in aqueous solutions with a pH of about 6.8 and greater. Most preferably, the mixture of hydroxypropyl methylcellulose acetate succinate is comprised of about 54% by weight hydroxypropyl methylcellulose acetate succinate that is soluble in aqueous solutions with a pH of about 5.5 and greater and about 7.7% by weight hydroxypropyl methylcellulose acetate succinate that is soluble in aqueous solutions with a pH of about 6.8 and greater.

Preferably, each of the one or more tablets has a weight of about 20 mg, and preferably comprises about 7.5 mg cyclobenzaprine hydrochloride. Preferably, the capsule of the composition contains two or four tablets.

In a further aspect of the present invention, there is provided a process for the manufacture of an extended-release cyclobenzaprine capsule comprising:
(1) preparing one or more matrix-type tablets by:
   (a) dispensing the cyclobenzaprine, or a pharmaceutically acceptable salt thereof, one or more polymers that alone or in admixture provide extended-release in an aqueous media, and optionally, one or more other pharmaceutically acceptable excipients;
   (b) mixing the dispensed materials to form a resulting mixture;
   (c) compacting the resulting mixture to form granules; and
   (d) milling the granules;
(2) optionally adding and mixing one or more other pharmaceutical excipients to the granules;
(3) compressing the granules to form the one or more tablets; and
(4) using the one or more tablets to fill the capsule, without applying an extended release coating to the tablets.

In a preferred embodiment of the process, the granules are compressed into tablets on a tablet press using 0.075″ to 0.250″ diameter round tooling, more preferably with about 0.125″ diameter round tooling.

In a further preferred embodiment of the process, each of the one or more tablets has a weight of from about 10 mg to about 30 mg, and more preferably of about 20 mg.

In a further preferred embodiment of the process, each of the one or more tablets contains about 7.5 mg of cyclobenzaprine hydrochloride.

In a further preferred embodiment of the process, each of the one or more tablets is comprised of about 35% to about 40% by weight cyclobenzaprine hydrochloride, about 55% to about 70% by weight hydroxypropyl methylcellulose acetate succinate, about 0.3% to about 0.9% by weight magnesium stearate, and about 0.05% to about 0.5% by weight colloidal silicon dioxide. More preferably, each of the one or more tablets is comprised of about 37.5% by weight cyclobenzaprine hydrochloride, about 61.7% by weight hydroxypropyl methylcellulose acetate succinate, about 0.6% by weight magnesium stearate, and about 0.2% by weight colloidal silicon dioxide. Even more preferably, the hydroxypropyl methylcellulose acetate succinate is a mixture of hydroxypropyl methylcellulose acetate succinate grades wherein one grade of hydroxypropyl methylcellulose acetate succinate is soluble in aqueous solutions with a pH of about 5.5 and greater, and a second grade of hydroxypropyl methylcellulose acetate succinate is soluble in aqueous solutions with a pH of about 6.8 and greater. Most preferably, the mixture of hydroxypropyl methylcellulose acetate succinate is comprised of about 54% by weight hydroxypropyl methylcellulose acetate succinate that is soluble in aqueous solutions with a pH of about 5.5 and greater and about 7.7% by weight hydroxypropyl methylcellulose acetate succinate that is soluble in aqueous solutions with a pH of about 6.8 and greater.

In a further preferred embodiment of the process, the capsule contains two or four tablets.

### DETAILED DESCRIPTION OF THE INVENTION

Although extended-release formulations of cyclobenzaprine are known, most notably, as capsules containing beads with an extended-release coating in the currently marketed AMRIX® capsules, there is a need for new extended-release formulations that address the manufacturing complexity associated with the manufacture of such AMRIX® capsules, including, in particular, the need to apply an extended release coating. The new extended-release cyclobenzaprine formulations of the present invention provide capsules that, preferably, are bioequivalent to the currently marketed AMRIX® product, but that have the advantage of a simplified manufacturing process.

In the dosage form of the present invention, each capsule is filled with one or more tablets. Each of the one or more tablets present in the capsule comprises cyclobenzaprine, or a pharmaceutically acceptable salt thereof, in a polymer matrix that will provide extended release of the cyclobenzaprine in aqueous media, specifically in the gastrointestinal tract. Preferably, the release profile of the one or more tablets is adapted such that the capsule of the present invention is bioequivalent to the marketed AMRIX® capsules. Thus, in a preferred embodiment, the present invention provides an improved dosage form that maintains bioequivalence with the currently marketed AMRIX® product, but that offers a more favourable manufacturing process that does not require the preparation of a multi-particulate dosage form where the individual beads have one or more coatings applied to them.

Each capsule of the present invention contains one or more matrix-type tablets as opposed to the multitude of coated extended release beads of the currently marketed AMRIX® capsules. The preferred capsule contains a total of either 15 mg or 30 mg of cyclobenzaprine hydrochloride, or a therapeutically equivalent dose of cyclobenzaprine or a pharmaceutically acceptable salt thereof, to correspond with the strengths of the commercially available AMRIX® extended release capsules containing cyclobenzaprine hydrochloride. For ease of manufacturing, it is further preferred that the one or more tablets each contains an amount of cyclobenzaprine or a pharmaceutically acceptable salt thereof that allows these tablets to be used in making either a 15 mg strength capsule or a 30 mg strength capsule. It is thus preferred that each of the one or more tablets contains either about 15 mg, 7.5 mg, 5 mg, 3.75 mg, 3 mg or 2.5 mg cyclobenzaprine hydrochloride or a therapeutically equivalent amount of cyclobenzaprine, or a pharmaceutically acceptable salt thereof. The total weight of each of the one or more tablets will preferably be from about 5 mg to about 50 mg, and more preferably from about 8 mg to about 40 mg.

Most preferably each of the one or more tablets contains about 7.5 mg cyclobenzaprine hydrochloride, which allows for capsules containing a total of about 15 mg and about 30 mg cyclobenzaprine hydrochloride to be prepared with two and four tablets, respectively.

Each of the one or more tablets comprises of a matrix comprising cyclobenzaprine hydrochloride and a polymer or mixture of polymers that will provide extended release in aqueous media. The polymer or mixture of polymers will preferably be either water-insoluble polymers or enteric polymers, which will be understood to mean polymers that are insoluble in aqueous media at pH up to at least about 4.5, so as not to be soluble in gastric fluid, but are soluble at higher pH, so as to be soluble in intestinal fluid. Inclusion of an enteric polymer in the one or more tablets is preferred. Optionally, the matrix may also include other excipients that do not contribute, or do not contribute significantly, to the extended release properties provided by the polymers.

The one or more tablets and the capsules containing the one or more tablets of the present invention, will preferably, when dissolution tested using USP Apparatus 2, exhibit a release profile substantially corresponding to the following pattern:
after 2 hours in 900 mL of 0.1 N HCl, at 37° C. at 50 rpm, no more than about 40% by weight of the total cyclobenzaprine is released, and
after 8 hours in 900 mL of 0.05 M phosphate buffer at pH 6.5, 37° C. and 75 rpm, at least about 60% by weight of the total cyclobenzaprine is released.

The composition of the tablets, and in particular the identity and quantity of the polymer, is selected to enable these dissolution specifications to be met by the tablets without the need to apply to the one or more tablets an extended release coating comprising a water insoluble polymer. Accordingly, the one or more tablets will preferably be without an extended release coating, and more preferably, will be uncoated.

Suitable water insoluble polymers for use in preparing the dosage forms of the present invention include, for example, ethylcellulose, cellulose acetate, polyvinyl acetate, neutral copolymers based on ethylacrylate and methylmethacrylate, copolymers of acrylic and methacrylic acid esters with quaternary ammonium groups, and ammonio methacrylic acid copolymers.

Suitable enteric polymers for use in preparing the dosage forms of the present invention include, for example, methacrylic acid copolymers, polyvinyl acetate phthalate, and hydroxypropylmethylcellulose acetate succinate (also known as hypromellose acid succinate and HPMCAS). Most preferably, the enteric polymer is HPMCAS. HPMCAS is sold by, for example, Shin-Etsu Chemical Co., Ltd. under the tradename AQOAT®, and is offered in three grades, herein referred to as HPMCAS-LF, HPMCAS-MF and HPMCAS-HF. HPMCAS-LF is soluble in aqueous solutions with a pH of about 5.5 and greater; HPMCAS-MF is soluble in aqueous solutions with a pH of about 6.0 and greater; and HPMCAS-HF is soluble in aqueous solutions with a pH of about 6.8 and greater.

The tablets will preferably comprise from about 10% to about 60% of cyclobenzaprine hydrochloride by weight, and more preferably from about 25% to about 50% of cyclobenzaprine hydrochloride by weight.

The tablets will preferably comprise from about 20% to about 80% of one or more polymers by weight, and more preferably from about 40% to about 70% of one or more polymers by weight.

The tablets will preferably comprise from about 20% to about 80% of one or more enteric polymers by weight, and more preferably from about 40% to about 70% of one or more enteric polymers by weight.

The invention will be better understood from the following example, which is intended to be illustrative of the invention and not limiting. It will be apparent to the skilled reader that various alterations may be made when using the compositions and methods of the present invention without departing from the scope or intent thereof.

### Example 1

Ingredients for a 26 kg batch were dispensed as follows:

| | |
|---|---|
| Cyclobenzaprine HCl | 9.75 kg |
| HPMCAS-LF | 14.04 kg |
| HPMCAS-HF | 2.00 kg |
| Magnesium Stearate | 0.156 kg |
| Colloidal Silicon Dioxide | 0.052 kg |
| Total | 26.0 kg |

The cyclobenzaprine HCl, HPMCAS-LF, HPMCAS-HF, one-third of the magnesium stearate and one-half of the colloidal silicon dioxide were mixed, compacted and milled. After milling, the remainder of the magnesium stearate and colloidal silicon dioxide were then added, and the material was mixed.

This final mixture was then compressed into tablets on a tablet press using 0.125″ diameter tooling to provide an average weight per tablet of 20 mg. Each tablet thus contained approximately 7.5 mg of cyclobenzaprine hydrochloride.

Capsules of size #4 were filled with 4 tablets per capsule, to give capsules each comprising 30 mg of cyclobenzaprine hydrochloride.

The above capsules, when dissolution tested using USP Apparatus 2, were found to comply with the following specifications:
After 2 hours in 900 mL of 0.1 N HCl, at 37° C. at 50 rpm, no more than about 40% by weight of the total cyclobenzaprine is released; and
After 8 hours in 900 mL of 0.05 M phosphate buffer at pH 6.5, at 37° C. and 75 rpm, at least about 60% by weight of the total cyclobenzaprine is released.

The results of the dissolution testing were as follows:

0.1N HCl at 50 rpm

| | % Released (avg. or |
|---|---|
| Time (min) | three capsules) |
| 30 | 8.6 |
| 60 | 12.6 |
| 90 | 15.8 |
| 120 | 18.6 |

0.05M Phosphate Buffer at pH 6.5 at 75 rpm

| | % Released (avg. or |
|---|---|
| Time (hrs) | three capsules) |
| 2 | 21.4 |
| 4 | 54.5 |
| 6 | 76.1 |
| 8 | 92.8 |

Bioequivalence studies were also performed on the above capsules versus commercially available 30 mg AMRIX® capsules.

In a comparative bioavailability study in 21 subjects in the fasted state, the mean results, as a ratio of the parameters for the capsule of Example 1 and the AMRIX® capsule, were:

| | |
|---|---|
| AUC_{(0-144 h)} ratio | 110.6% |
| Cₘₐₓ ratio | 102.9% |

In a comparative bioavailability study in 39 subjects in the fed state, the mean results, as a ratio of the parameters for the capsule of Example 1 and the AMRIX® capsule, were:

| | |
|---|---|
| AUC_{(0-144 h)} ratio | 103.1% |
| Cₘₐₓ ratio | 99.2% |

Based the results of the bioequivalence studies, the above capsules were considered to be bioequivalent to the commercially available AMRIX® capsules.

1. An extended-release composition of cyclobenzaprine for oral administration in the form of a capsule containing one or more tablets, wherein each of the one or more tablets is a matrix-type tablet that comprises cyclobenzaprine, or a pharmaceutically acceptable salt thereof, one or more polymers, and optionally, one or more other pharmaceutically acceptable excipients, that provides extended release in an aqueous media without an extended release coating on the tablets;
wherein the capsule, when dissolution tested using USP Apparatus #2, complies with the following specifications:
(1) after 2 hours in 900 mL of 0.1 N HCl, at 37° C. at 50 rpm, no more than about 40% of the cyclobenzaprine, or pharmaceutically acceptable salt thereof, is released from the composition; and
(2) after 8 hours in 900 mL of 0.05 M phosphate buffer at pH 6.5, 37° C. and 75 rpm, at least about 60% of the cyclobenzaprine, or pharmaceutically acceptable salt thereof, is released from the composition.

2. The composition of claim 1, wherein the one or more polymers comprises at least one polymer that is at least one of water-insoluble and an enteric polymer.

3. The composition of claim 1, wherein the one or more polymers comprises at least one enteric polymer.

4. The composition of claim 3, wherein the polymer is hydroxypropyl methylcellulose acetate succinate.

5. The composition of claim 4, wherein the polymer is a mixture of low and high pH-dissolving hydroxypropyl methylcellulose acetate succinate.

6. The composition of claim 3, wherein the tablets comprise from about 25% to about 50% by weight cyclobenzaprine, or a pharmaceutically acceptable salt thereof, and from about 40% to about 70% by weight hydroxypropyl methylcellulose acetate succinate.

7. The composition of claim 6, wherein the hydroxypropyl methylcellulose acetate succinate is a mixture of low and high pH-dissolving hydroxypropyl methylcellulose acetate succinate.

8. The composition of claim 1, wherein the weight of each tablet is from about 5 mg to about 50 mg.

9. The composition of claim 8, wherein the total amount of cyclobenzaprine hydrochloride in each of the one or more tablets is from about 2.5 mg to about 15 mg.

10. The composition of claim 9, wherein the total amount of cyclobenzaprine hydrochloride in each of the one or more tablets is at least one of the following: 15 mg, 7.5 mg, 5 mg, 3.75 mg, 3 mg, and 2.5 mg.

11. The composition of claim 1, wherein the one or more tablets are comprised of about 35% to about 40% by weight cyclobenzaprine hydrochloride, about 55% to about 70% by weight hydroxypropyl methylcellulose acetate succinate, about 0.3 to about 0.9% by weight magnesium stearate, and about 0.05 to about 0.5% by weight colloidal silicon dioxide.

12. The composition of claim 11, wherein the one or more tablets are comprised of about

37. 5% by weight cyclobenzaprine hydrochloride, about 61.7% by weight hydroxypropyl methylcellulose acetate succinate, about 0.6% by weight magnesium stearate, and about 0.2% by weight colloidal silicon dioxide.

13. The composition of claim 12, wherein the hydroxypropyl methylcellulose acetate succinate is a mixture of hydroxypropyl methylcellulose acetate succinate grades, wherein one grade of hydroxypropyl methylcellulose acetate succinate is soluble in aqueous solutions with a pH of about 5.5 and greater, and a second grade of hydroxypropyl methylcellulose acetate succinate is soluble in aqueous solutions with a pH of about 6.8 and greater.

14. The composition of claim 13, comprising about 54% by weight hydroxypropyl methylcellulose acetate succinate that is soluble in aqueous solutions with a pH of about

5. 5 and greater and about 7.7% by weight hydroxypropyl methylcellulose acetate succinate that is soluble in aqueous solutions with a pH of about 6.8 and greater.

15. The composition of claim 14, wherein each of the one or more tablets has a weight of about 20 mg.

16. The composition of claim 11, wherein the capsule contains at least one of two tablets and four tablets.

17. A process for the manufacture of an extended-release cyclobenzaprine capsule of claim 1 comprising:
(1) preparing one or more matrix-type tablets by:
(a) dispensing the cyclobenzaprine, or a pharmaceutically acceptable salt thereof, one or more polymers that alone, or in admixture, provide extended-release in an aqueous media, and optionally, one or more pharmaceutically acceptable excipients;
(b) mixing the dispensed materials to form a resulting mixture;
(c) compacting the resulting mixture to form granules; and
(d) milling the granules;
(2) optionally adding and mixing one or more pharmaceutical excipients to the granules;
(3) compressing the granules to form the one or more tablets; and
(4) using the one or more tablets to fill the capsule, without applying an extended release coating to the tablets.

18. The process of claim 17, wherein the granules are compressed into tablets on a tablet press using 0.075 inch to 0.250 inch diameter round tooling.

19. The process of claim 17, wherein each of the one or more tablets is comprised of about 35% to about 40% by weight cyclobenzaprine hydrochloride, about 55% to about 70% by weight hydroxypropyl methylcellulose acetate succinate, about 0.3% to about 0.9% by weight magnesium stearate, and about 0.05% to about 0.5% by weight colloidal silicon dioxide.

20. The process of claim 19, wherein the hydroxypropyl methylcellulose acetate succinate is a mixture of hydroxypropyl methylcellulose acetate succinate grades, wherein one grade of hydroxypropyl methylcellulose acetate succinate is soluble in aqueous solutions with a pH of about 5.5 and greater, and a second grade of hydroxypropyl methylcellulose acetate succinate is soluble in aqueous solutions with a pH of about 6.8 and greater.
